(19) United States
(12) Patent Application Publication
(10) Pub. No.: US 2017/0020999 A1
(43) Pub. Date: Jan. 26, 2017

(54) Composition for prevention and/or treatment of chronic inflammation and concomitant biofilms in the gastrointestinal tract

(71) Applicant: BIOTECHNOBEL SA, Brussels (BE)

(72) Inventor: Seyed Saied BATHAEI, Brussels (BE)

(21) Appl. No.: 15/301,840

(22) PCT Filed: Apr. 3, 2015
(86) PCT No.: PCT/EP2015/097022

§ 371 Date: Oct. 4, 2016

(30) Foreign Application Priority Data
Apr. 4, 2014 (EP) 14163644.9

(51) Int. Cl. A61K 38/54; A61K 31/198; A61K 31/07; A61K 31/59; A61K 33/30; A61K 31/722; A61K 36/53; A61K 31/519; A61K 31/4415; A61K 31/375; A61K 31/714; A61K 33/06; A61K 33/44; A61K 36/22; A23L 33/165; A23L 33/175; A23L 33/155; A61K 47/42

(52) U.S. Cl. CPC A61K 38/54; A61K 47/42; A61K 31/198; A61K 31/07; A61K 31/59; A61K 33/30; A61K 31/722; A61K 36/53; A61K 31/519; A61K 31/4415; A61K 31/375; A61K 31/714; A61K 33/06; A61K 33/44; A61K 36/22; A23L 33/165; A23L 33/175; A23L 33/155; A23V 2002/00

(57) ABSTRACT

A pharmaceutical or neutraceutical composition for preventing or treating of chronic inflammation and concomitant biofilms in the gastrointestinal tract (GI tract). The multidimensional clinically tested composition accelerates intestinal epithelium recovery and it destabilises Gram-negative bacteria in their habitat, but it also eliminates their LPS molecules, which are highly allergenic. The synergistic composition contains a pharmaceutically effective amount of at least L-glutamine with zinc and vitamine A for recovery of the epithelium cells of the gastrointestinal tract, at least enzymes selected from the group of polysaccharidases, proteases, lipases and/or antioxidants, for decomposing a bio film that is present in the gut, at least one chelator for inorganic components such as iron and at least one binder for organic components originating from the decomposition of the biofilm and/or bacteria.

The present invention relates to a pharmaceutical or neutraceutical composition for prevention and/or treatment of chronic inflammation in the presence of concomitant biofilms in the gastrointestinal tract (GI tract). Often biofilms, chronic inflammation of the GI tract and/or dysbiosis in the GI tract are interrelated.

Dysbiosis refers to a state of imbalance among the gut flora inside the human body. The gut flora, or also called intestinal micro-flora, consists of a complex of microorganism species, including bacteria, yeast, fungi, viruses and parasites, that live in the digestive tract. Eubiosis, in contrast to dysbiosis, refers to a healthy balance of the micro-flora in the gastrointestinal tract.

The intestinal flora is essential for basic biological mechanisms required for human and animal life, such as digestion, energy production and detoxification.

Bacteria make up most of the flora in the colon and up to 60% of the dry mass of faeces. About 300 to 1000 different species live in the gut of which probably about 30 to 40 species represent 99% of the bacterial population.

Many chronic disorders come from digestive problems and inadequate nutrient absorption. Proper gastrointestinal function is needed to eliminate toxic substances, pathogenic microbes, and undigested food particles from the body in order to prevent health problems. Nutrients require a specific internal environment to be properly digested and transported throughout the body.

Abnormal intestinal microorganisms in the GI tract are widely known to cause disease. Research shows a relationship between the GI tract and the neurological, hepatic, and immune systems.

Eubiosis can vary from individual to individual. Table 1 shows an example of a flora with typical reference values for a state of eubiosis as measured and analysed in stool. Table 1 shows typical population of normal bacteria in stool. Table 2 furthers shows typical reference values of other factors, which can be measured in stool for a state of eubiosis, i.e. intestinal immune function, overall intestinal health, and inflammation markers.

For restoring and/or maintaining a state of eubiosis most often it is suggested to administer probiotics and/or prebiotics. The international patent application WO 2013/037068 A1 further suggests the use of a synthetic stool preparation comprising a mixture of bacterial strains for treating disorders of the GI tract, i.e. dysbiosis.

TABLE 1
Typical reference values of microorganisms, which can be measured
in stool for a state of eubiosis

| Aerobic bacterial micro-flora | CFU/g* | |
|---|---|---|
| Escherichia coli | 5.10⁵-10⁷ | CFU/g |
| Enterobacteriaceae | ≦9.10⁴ | CFU/g |
| Proteus mirabilis | ≦9.10⁴ | CFU/g |
| Proteus vulgaris | ≦9.10⁴ | CFU/g |
| Klebsiella oxytoca | ≦9.10⁴ | CFU/g |
| Klebsiella pneumoniae | ≦9.10⁴ | CFU/g |
| Citrobacter spp. | ≦9.10⁴ | CFU/g |
| Serratia spp. | ≦9.10⁴ | CFU/g |
| Hafnia alvei | ≦9.10⁴ | CFU/g |
| Morganella morganii | ≦9.10⁴ | CFU/g |
| Providencia spp. | ≦9.10⁴ | CFU/g |
| Pseudomonas spp. | ≦9.10⁴ | CFU/g |
| Enterococcus | 1.10⁶-1.10⁷ | CFU/g |
| Streptococcus β hemolytic | ≦9.10⁴ | CFU/g |
| Bacillus spp. | ≦9.10⁴ | CFU/g |
| Staphylococcus aureus | ≦9.10⁴ | CFU/g |
| | CFU/g | |
| Anaerobic bacterial micro-flora | | |
| Bacteroides spp. | 1.10⁸-1.10¹⁰ | CFU/g |
| Clostridium spp. | 1.10⁵ | CFU/g |
| Bifidobacterium spp. | 1.10⁸-1.10¹⁰ | CFU/g |
| Lactobacillus spp. | 1.10⁵-1.10⁸ | CFU/g |
| Salmonella spp. | negative | |
| Other micro-flora | | |
| Candida albicans | ≦1.10³ | CFU/g |
| Geotrichum | ≦1.10³ | CFU/g |
| Molds | ≦1.10³ | CFU/g |

*CFU/g, stool samples are collected and plated onto selective media to determine the amount of colony-forming unit (CFU) per gram of stool; the method and type of selective medium is known as such.

When, in dysbiosis, the intestinal flora is in imbalance the metabolic activity of the intestinal flora changes and the presence of potential pathogenic microorganisms increases.

This leads to the liberation of metabolites, which are potentially toxic, e.g. endotoxins. As an example, lipopolysaccarides (LPS) are part of the cell wall of Gram-negative bacteria, which also play a role in the building of biofilms (1-2). These metabolites or endotoxins are extremely virulent and lead to a broad range of diseases, which are called degenerative chronic diseases. Examples are irritable bowel syndrome, inflammatory bowel disease, rheumatoid arthritis, ankylosing spondylitis, multiple sclerosis, Parkinson, chronic fatigue, eczema, food allergy, some forms of cancer, metabolic syndrome, artherosclerosis etc. (3).

TABLE 2
Typical reference values of some immunity markers, which can be
measured in stool for a state of eubiosis*

| pH | 5.8-6.5 | |
|---|---|---|
| Pancreatic elastase 1 | ≧200 | μg/g |
| Immunology | | |
| Immunoglobulin A (sIgA) | 510-2040 | μg/ml |
| Calprotectin | ≦50 | mg/kg |
| Alpha-1-antitrypsin (α₁AT) | ≦270 | μg/ml |

*Pancreatic elastase 1, Calprotectin and sIgA are determined by enzyme-linked immunosorbent assays (ELISA) which are known as such

Gut dysbiosis can lead to changes in the lining of the bowel that increase the permeability of the intestine, resulting in leaky gut syndrome and chronic inflammation of the GI tract. The intestinal lining is a barrier that normally only allows properly digested fats, proteins, and carbohydrates to pass through and enter the bloodstream. When the intestinal lining gets battered, by e.g. bacterial toxins, the intestinal lining loses its integrity. This opens the way to let in bacteria, viruses, parasites and even undigested food macromolecules. These will activate the immune system and often hyper-stimulate it so that it squirts out inflammatory substances called cytokines and act to weaken the intestinal wall. This leads to chronic inflammation and from there to a whole range of diseases. The agitated immune system may also become so unstable such that it results in autoimmune diseases.

Often gut dysbiosis leads to the formation of biofilms by pathogenic bacteria. These biofilms protect the pathogenic bacteria resulting in chronic infections that are difficult to eradicate. In particular, Gram-negative bacteria have a high tendency to make biofilms.

The human body, with its wide range of moist surfaces and mucosal tissue, is an excellent place for biofilms to thrive. Bacteria present in a biofilm have a significantly greater chance of evading the battery of immune system cells, which more easily attack planktonic microbial cells. The bacterial biofilms seem to have great potential for causing human disease such as common infections as urinary tract infections caused by E. coli and other pathogens, catheter infections caused by Staphylococcus aureus and other Gram-positive pathogens, common dental plaque formation and gingivitis (4-6).

There is a ‘biofilm cycle’, starting from attachment of bacteria to colony formation, biofilm formation, maturation and release of bacteria. This can be a cycle of weeks or even months. The acute inflammation stage renders it impossible for the immune system to solve the problem and to eliminate the infection.

In an effort to target bacterial biofilms, high, constant doses of antibiotics are administered to patients. Unfortunately, even when administered in high doses, antibiotics may only temporarily weaken the biofilm, but are mostly incapable of completely destroying the biofilm. Inevitably a number of bacterial cells persist in the remaining biofilm and allow the biofilm to regenerate to strength. Often the regenerated biofilm will contain bacteria with improved resistance.

The international patent application WO 2011/063394 A1 proposes an interesting composition for the elimination of biofilms in e.g. the gastrointestinal tract. The proposed composition contains mainly enzymes for breaking down the biofilm. However, it does not provide a solution for preventing a subsequent fast regeneration of a new bio film, which is likely to occur after breaking down of the biofilm.

The gut dysbiosis hypothesis suggests that a number of factors associated with modern living have a detrimental impact on the micro-flora of the gastrointestinal tract. Factors such as antibiotics, psychological and physical stress, certain dietary components such as food additives (such as ‘azo’ colorants, or certain flavors), and chemical products such as pesticides, herbicides, fungicides or insecticides have been found to contribute to intestinal dysbiosis (7-8).

The longer we live, the higher the probability that such an accident will occur, leading to a persistent state of dysbiosis.

The present invention aims to provide a pharmaceutical or nutraceutical composition for prevention and/or treatment of chronic inflammation of the gastrointestinal tract (GI tract) and/or biofilms in the GI tract, which are often associated to e.g. dysbiosis. The treatment of biofilms includes disruption and removal of the biofilm in the GI tract and further prevention of reinstatement of the biofilm. The invention further aims to subsequently restore eubiosis and a healthy intestinal epithelium (enterocyte) with normal permeability.

To this end, the present invention provides a pharmaceutical composition for preventing and/or treating bio films in the gastrointestinal tract, for eliminating pathogenic bacteria and their allergenic corpses, i.e. debris, as well as their endotoxins, and further for restoring the gastrointestinal epithelium (enterocyte) and for repopulating the GI tract with a beneficial flora, referred to as eubiosis, as set out in the annexed claims.

As such the composition according to the invention comprises

(i) enzymes selected from the group of polysaccharidases, proteases, lipases and/or antioxidant enzymes, for decomposing a biofilm that is present in the gut;

(ii) at least one chelator, comprising lactoferrin, for binding inorganic components such as iron; and

(iii) at least one binder, for binding organic components originating from the decomposition of the bio film and/or bacteria;

(iv) glutamine, vitamin A, vitamin D and zinc, for facilitating recovery of mucosa cells of the gut.

According to an interesting embodiment the composition further comprises chitosan, for an antibacterial activity against Gram-negative bacteria.

According to another interesting embodiment the composition further comprises Rosmarinus officinalis extract, for reducing activity and adhesion of the bio film.

According to a further interesting embodiment the composition comprises at least one binder for binding organic components originating from the decomposition of the bio film and at least one binder for binding organic components originating from the decomposition of bacteria of the bio film.

According to a preferred further interesting embodiment the composition comprises
at least one binder for binding organic components originating from the decomposition of the biofilm selected from the group of inositol and/or rice bran or a combination thereof;
at least one binder for binding organic components originating from the decomposition of bacteria of the biofilm selected from the group of Punica granatum extract, Citrus aurantium extract, Quercus rubra extract and/or Quercus petraea extract, or a combination thereof; and
at least one binder for binding organic components originating from the decomposition of bacteria of the biofilm selected from the group of phosphatidylcholine and/or magnesium or a combination thereof;

The composition may also contain further interesting compounds such as anti-inflammatory ingredients, further vitamins, activated carbon and/or Pistacia lentiscus resin.

According to the invention the ingredients of the composition may be offered as food supplement, functional food ingredients or as pharmaceutical ingredients in order to prepare a composition for oral administration for preventing and/or treating biofilms in the GI tract and/or chronic inflammation of the GI tract.

Particularities and advantages of the invention will become clear from the following description and practical embodiments of the composition of the invention; the description and practical embodiments are given as example only and do not limit the scope of the claimed protection in any way.

The present invention proposes a synergistic composition for oral administration to treat chronic inflammation of the gastrointestinal tract and to destabilize bio films, that include and protect harmful microorganisms, in the intestinal tract and further to subsequently restore normal gut flora and the intestinal epithelium (enterocyte). Besides the destabilisation and disruption of the biofilm, in order to reduce chronic inflammation of the gastrointestinal tract, the composition according to the invention enables the immune system to be restored (such as secretory immunoglobulin A: sIgA and intestinal alkaline phosphatase) and hence to take control of the bacterial flora.

The complexity of the composition can impose to administer the components via separate blends or capsules. As an example, polysaccharidases should preferably not be blended with their substrate such as e.g. rice bran. The possible incompatibility of different ingredients of the composition is known as such for the person skilled in the art.

The composition, according to the invention, is a multidimensional and, hence, synergistic composition for destabilizing and disrupting bio films and restoring a state of eubiosis, which acts on three different fields:

I. disrupting the bio film;

II. decreasing pathogenic bacteria by destruction of the cell wall, especially Gram-negative bacteria; and

III. accelerating intestinal epithelium recovery.

When the bio film has been eliminated it will be useful to further create the conditions for a balanced intestinal flora in the GI tract for obtaining a stable state of eubiosis. Conditions for restoration of the colonic epithelium and enhancing a balanced flora are known from e.g. European patent application EP 2 478 779 A1. However, only providing optimal conditions for eubiosis to move the bacterial intestinal flora back into balance will fail to maintain a stable state of eubiosis when a bio film is present.

I. Disrupting the Biofilm

Creating the right conditions for a healthy bacterial flora sometimes does not suffice long term if pathogenic bacteria, especially the Gram-negative bacteria, hide in a bio film. If there is an excess of pathogenic bacteria, there is a high probability of biofilm formation, since a lot of these pathogenic bacteria have the tendency to build biofilms. In this invention, a series of natural ingredients are used to destabilize and disrupts these biofilms.

The composition of the bio film essentially consists of exopolysaccharide, i.e. polysaccharides, homopolysaccharides and heteropolysaccharides, organic substituents such as acyl, and inorganic substituents such as phosphate or sulphate. Recently, it was found that there are also biofilm associated proteins.

The bio film is preferably at least partially detached from the intestinal wall by an anti-adhesive effect of Rosmarinus officinalis extract.

Rosmarinus officinalis extract also prevents the formation of a new bio film by Gram-negative bacteria that are escaped from the bio film.

The biofilm is then attacked by a range of enzymes and by taking away essential nutrients for the building of biofilms. Hence, enzymes and chelators are used in the composition in order to destabilize the bio film. The destabilized bio film then leads to liberation of pathogenic bacteria, especially, Gram-negative bacteria, which are present in the biofilm. After liberation of Gram-negative bacteria from the bio film it will be easier to attack and destroy these potential pathogenic bacteria.

The exopolysaccharides are attacked and disrupted with polysaccharidase enzymes such as e.g.: alpha-amylase, beta-amylase, glucosamylase, alpha-galactosidase, invertase, maltase, cellulose, hemicellulose, xylanase, pectinase, pectinesterase, pullulanase, and/or dextranase.

The protein component is hydrolysed by proteases such as e.g.: bromelain, papain, ficin, and/or other proteases preferably from plant origin.

The bond esters and acyl groups are hydrolysed by lipolytic enzymes such as e.g.: lipases and phospholipases.

The inorganic substituents, necessary for building biofilms, are neutralized by complexation with chelators and binders such as e.g. inositol, rice bran and/or lactoferrin. Iron is essential for the activity of microorganism, especially Gram-negative bacteria, which builds biofilms. Iron can be neutralised by e.g. lactoferrin.

II. Decreasing Pathogenic Bacteria and Immunogenic Substances

Potential pathogenic bacteria and also immunogenic substances origination from these bacteria, which are released from the disrupted bio film, need to be neutralised in order to prevent exposing the intestinal flora to high amounts of potential pathogenic bacteria and/or to prevent a too excessive immune response.

Breaking down the bio film will indeed inevitably result in a high risk of e.g. life-threatening inflammatory reactions, excessive immune response, diarrhea, endotoxemia and/or septic shock by the release of endotoxins.

The organic components origination from the decomposition of the bio film are neutralized for evacuation by complexation with binders such as inositol and rice bran.

The release of bacteria from the bio film will subsequently lead to an intestinal micro-flora that is vulnerable for moving to a state of dysbiosis. The state of dysbiosis will again increase the risk to the formation of a bio film.

Antibiotics could be used for decreasing the presence of pathogenic bacteria, but they also cause a lot of damage to the good bacterial flora such that restauration of the good bacterial flora gets much more difficult.

Lipopolysaccharide (LPS), the major component of endotoxin, is present in the outer membrane of Gram-negative bacteria and triggers immune response by interacting with LPS receptors on the surface of immune cells (5). Too much endotoxin release in the presence of an overwhelming Gram-negative bacterial infection can contribute to life-threatening inflammatory reactions, excessive immune response, diarrhea, endotoxemia and/or septic shock. LPS also plays an important role in suppression of the activity of lipoprotein lipase (LPL). This leads to hypertriglyceridemia.

LPS of the cell wall of Gram-negative bacteria is preferably attacked in a systematic way by one or several components selected from the group of e.g.: polysaccharidase enzymes; proteolytic enzymes; lipolytic enzymes; magnesium; polyphenols; tannins; chitosan; lactoferrin; EDTA; and/or activated carbon.

Polysaccharidase enzymes attack and hydrolyse the polysaccharide parts of the LPS consisting of the O-antigen, the outer core and the inner core. Suitable polysaccharidases are alpha-amylase, beta-amylase, amyloglucosidase, alpha-galactosidase, invertase, maltase, cellulase, lactase, hemicellulase, pectinase, xylanase, dextranase, and/or pullulanase.

Proteolytic enzymes hydrolyse the protein part of the bacterial cell wall. Different types of proteases such as plant proteases/cysteine proteases, e.g. bromelain, papain and/or ficin are suitable.

Lipolytic enzymes hydrolyse the lipid A of the LPS. Lipid A is responsible for toxicity of Gram-negative bacteria and is a very potent stimulant of the immune system. According to the present invention, it is recommended to eliminate lipid A for protection of the immune system and for preventing an excessive immune reaction.

In the composition according to the invention, proteases are preferably always combined with lipase for attacking the LPS of the cell wall of Gram-negative.

Magnesium binds to the phosphate part of the inner core (phospholipid), thereby neutralizing it.

Polyphenols that are present in plant extracts such as e.g. Punica granatum extract (pomegranate), Rosmarinus officinalis extract (rosemary), Citrus aurantium extract, Quercus rubra extract, and/or Quercus petraea extract bind the above polysaccharide parts, making them ready for evacuation through stool. These components are also well known for their anti-inflammatory and/or antioxidant effects. In case of dysbiosis, when the gut is chronically inflamed, the anti-inflammatory and/or antioxidant effects of the polyphenols in plant extracts are interesting features. Furthermore, these effects are also obtained by superoxide dismutase and catalase in the composition.

Rosmarinus officinalis extract is able to inhibit formation of the by bacterial biofilm by reducing its activity and its adhesion to the intestinal wall. Rosmarinus officinalis extract is also known as an antioxidant. It has been proven in numerous studies that Rosmarinus officinalis extract possesses a high free radical scavenging activity, but it also has a positive effect on maintaining lipid membrane stability. It has also been proven to be an effective antibiotic against many strains of bacteria.

Tannins, present in plant extracts such as e.g. Quercus rubra extract, Quercus petraea extract, and/or ellagitannin of Punica granatum, form complexes with proteins, starch, cellulose, polysaccharides and/or minerals. Tannin compounds have been found to interfere with bacterial adhesion by blocking LPS receptors. Tannins are naturally occurring plant polyphenols such as e.g. ellagitinin of Punica granatum, Quercus petraea and Quercus rubra. Their main characteristic is that they bind and precipitate proteins or that they form complexes with polysaccharides. Once precipitated, these components will be evacuated with the stools. Ellagitannin from Punica granatum possesses also an inhibitory effect on the LPS-induced inflammatory reaction (9). It has been shown that extracts of Punica granatum bark show considerable antibacterial activity for Gram-positive and Gram-negative bacteria (11). One of the most satisfactory definitions of tannins was given by Horvath (10): “Any phenolic compound of sufficiently high molecular weight containing sufficient hydroxyls and other suitable groups, i.e. carboxyls, to form effectively strong complexes with protein and other macromolecules under the particular environmental conditions being studied”.

Chitosan has antibacterial activity against, in particular, Gram-negative bacteria. As such, chitosan is an interesting optional component in the composition for selectively suppressing Gram-negative bacteria. Gram-negative bacteria compared to Gram-positive bacteria have better hydrophilicity and more negatively charged cell surfaces exhibiting greater interaction with chitosan. Accordingly, chitosan has preferable antibacterial activity against Gram-negative bacteria. Chitosan increases the permeability of the outer membrane and ultimately disrupts bacterial cell membranes. The damage is likely caused by the electrostatic interaction between NH₃⁺ groups of chitosan and carbonyl or phosphoryl groups of phospholipid components of cell membranes (12-13).

Lactoferrin binds free iron and affects bacterial membranes. The primary role of lactoferrin is to sequester free iron and, in doing so, to remove an essential substrate required for bacterial growth and bio film formation. The antibacterial action of lactoferrin is also explained by the presence of specific receptors on the cell surface of microorganisms. Lactoferrin binds to lipopolysaccharide (LPS) of bacterial cell walls, and the oxidized iron part of the lactoferrin oxidizes bacteria via formation of peroxides. This affects membrane permeability and results in cell lysis. Consequently, lactoferrin is an interesting component in the composition for its selective antibacterial activity and also its antibio film activity.

After brake up of LPS and liberation of the abundant anionic groups, lipid A and oligosaccharides, these fragments can be tightly bound by electrostatic interactions with divalent cations such as Mg²⁺. The negatively charged groups are selectively targeted by cationic Mg²⁺ and also by antimicrobial peptides.

Activated carbon absorbs harmful substances, e.g. toxins from the debris of Gram-negative bacteria, in the gastro-intestinal tract in order to eliminate and evacuate them with the faeces.

Hence, different components of the LPS are broken down, captured and evacuated by the faeces.

Addition of the Pistacia lentiscus resin decreases the colonic pH as a result of the production of organic acids by bacterial fermentation. The decrease in pH creates an environment that is both hostile to the survival of urease-producing gut flora, such as Klebsiella species (spp) and Proteus spp, and conducive to the growth of acid-resistant, non-urease-producing species, such as lactobacilli and bifidobacteria, resulting in reduced production of ammonia in the colonic lumen. In addition, acidification of colonic secretions reduces the absorption of ammonia by non-ionic diffusion.

Further, optionally providing prebiotic fibres, which are preferentially digested and used by the beneficial flora in the intestinal tract and which are known as such, will help with repopulation of the gut with a balanced intestinal flora in order to obtain a state of eubiosis in the GI tract. Providing prebiotic fibres and nutrients, which are preferentially digested and used by both human and the beneficial flora in the intestinal tract and which are known as such, will also stimulate the beneficial flora in the intestinal tract.

III. Intestinal Epithelium Recovery Restoring Immune Defense

Due to the presence of harmful microorganisms in the bio film and ensuing leaks in the gut, the immune system is hammered. Tight junctions are not intact anymore such that gut content leaks into the body, which results in an overcharging of the immune system.

Therefore the composition according to the invention treats the intestinal permeability with ingredients such as vitamin A, glutamine, zinc, Punica granatum extract, Curcuma longa extract, catalase, superoxide dismutase and vitamin B6. Another support for the immune system is the administration of anti-oxidants such as catalase, Punica granatum extract, Citrus aurantium extract, Rosmarinus officinalis extract, superoxide dismutase. Inflammation is a result of an immune reaction, and in itself it causes the immune system to react. This vicious circle needs to be broken down.

In the quest to fight bacterial overgrowth, natural allies from the immune system are key. The immune protection comes with a healthy intestinal wall.

Bacterial overgrowth can actually lead to even more damage of the protective endothelial lining of the gut. Bacteria produce their own enzymes, which destroy the protective mucus coat of the intestinal lining. Increased intestinal permeability is observed in association with several autoimmune diseases. Intestinal epithelium recovery is important for bringing the immune system back to normal, especially recovery of the intestinal alkaline phosphatase and secretory immunoglobulin A (sIgA). Several studies have shown that the intestinal alkaline phosphatase enzyme can dephosphorylate and detoxify the endotoxin component of LPS, likely through dephosphorylation of the lipid A moiety, the primary source of its endotoxic effects.

Intestinal alkaline phosphatase (IAP) is an intestinal brush border enzyme that is shown to function as a gut mucosal defense factor.

Inflammatory bowel disease is characterized by chronic inflammation of the intestine and is accompanied by damage of the epithelial lining and by undesired immune responses towards enteric bacteria. It has been demonstrated that intestinal alkaline phosphatase (IAP) protects against the induction of inflammation, possibly due to dephosphorylation of lipopolysaccharide (LPS). Consequently, the intestinal alkaline phosphatase contributes to the reduction of severe preserves the normal homeostasis of gut microbiota.

Previously, it was shown that intestinal alkaline phosphatase (IAP), a small intestinal brush border enzyme, functions as a gut mucosal defense factor limiting the translocation of gut bacteria to mesenteric lymph nodes.

Secretory IgA (sIgA) is the predominant class of antibody found in intestinal secretions. Although sIgA serves as the first line of defense in protecting the intestinal epithelium from enteric toxins and pathogenic microorganisms.

The intestinal permeability decreases the effect of mucosal epithelial barrier, as well as the activity of intestinal alkaline phosphatase (IAP) and secretory IgA (sIgA).

New epithelium is generated every 2 to 5 days. Therefore, providing proper nourishment to the cells of the small intestine is helpful for healing the lining of the gut. By restoring the intestinal epithelium, the initial antibacterial activity of sIgA and IAP are restored.

The regeneration of intestinal epithelium, including enterocytes, is, preferably, made by providing a combination of components selected from the group of e.g.: glutamine; Curcuma longa; Punica granatum extract; phosphatidylcholine; vitamin B9; vitamin A; vitamin C; vitamin D; vitamin B6; vitamin B12; zinc; magnesium; superoxyde dismutase (SOD); and/or catalase.

Glutamine, in particular L-glutamine, is the most important nutrient that gives support to the repair of the intestinal lining. It is the preferred fuel and nitrogen source for the small intestine. Glutamine improves intestinal epithelial cell functions, proliferation of the intestinal flora, as well as cellular differentiation and further reduces infections. Glutamine is an important energy source for the enterocytes, as for other cells with fast renewal rates, in particular, immune cells such as lymphocytes and macrophages. Glutamine controls stimulation and proliferation of the intestinal epithelial cells by their specific growth factor, the Epidermal Growth Factor I (EGF). It increases the effects of the growth factors responsible for cellular repair and proliferation.

Curcumin of Curcuma longa exhibits potent anti-inflammatory effects. Curcumin has antioxidant, antiviral and antifungal action. Curcumin is also a potent immunomodulator. An association between glutamine and curcumin is interesting in view of their complementary mechanistic properties, which correspond well to the pathological disturbances characterizing intestinal epithelial cell injury. Punica granatum extract is rich in punicalagin and exhibits potent anti-inflammatory effects on human intestinal epithelium. It could also be an interesting natural source contributing to prevent intestinal chronic inflammation.

Phosphatidylcholine, is an anti-inflammatory agent, and a surface hydrophobicity increasing compound with promising therapeutic potential in the treatment of inflammatory bowel disease.

Vitamin B9, also known as folic acid, folacin and/or folate, takes part in the good functioning of the intestinal mucous membrane.

Vitamin A supplements improves the intestinal inflammation. Two principal mechanisms, which seem to be involved in prevention of intestinal inflammation, are the effect of vitamin A on the immune system and the effect of vitamin A on the intestinal epithelial integrity. Retinol proves to be important for renewal of the epithelium and contributes to their maintenance. In partnership with zinc, it improves the intestinal permeability.

The inhibitory effects of vitamin D on colitis are known. Epidemiological studies have shown that low vitamin D status is common in Inflamatory Bowl Disease (IBD).

Vitamin C interacts with free radicals and can act as an antioxidant. It regenerates vitamin E, which is a protective antioxidant that is present in the cell membrane.

Vitamin B6 is involved in the metabolism of proteins, carbohydrate and lipids. It is a cofactor of several metabolic enzymes. Vitamin B6 is important for assimilation of magnesium and absorption of amino acids. It stimulates the immune system, it is important for the regulation of the tissues construction and it has an antioxidant activity. Vitamin B6 with zinc is needed to maintain intestinal wall integrity.

Zinc represents a capital nutrient of the intestinal mucous membrane. Zinc deficiency disturbs the total body growth and causes important reductions of the protein contents of the intestinal mucous membrane. Zinc plays an important role in the healing of tissues. It is a cofactor in many enzymatic systems, essential to protein synthesis, cellular proliferation, genetic expression of growth factors and steroid receptors. Zinc represents the last line of defence against oxidation of the sulfhydryl groups of the cellular membrane. Moreover, it inhibits bacterial lipase, decreases the intestinal hyperpermeability and increases the rate of prostaglandin E1 (PGE1) in the intestine, which is beneficial for the immune function.

Superoxide dismutase (SOD) and catalase are examples of suitable antioxidant enzymes that may be used in the composition according to the invention. The use of superoxyde dismutase (SOD) and catalase has beneficial effects on chronic inflammation of the colon. In addition, SOD supplements decrease the intestinal inflammation induced by pathogenic bacteria. The role of free radicals in certain gastro-intestinal disorders and inflammatory diseases of the intestine was shown by certain studies. Crohn's disease is characterized by the chronic inflammation of the gastro-intestinal mucous membrane. Several studies show the importance of anti-inflammatory action of SOD on the intestinal inflammation.

The composition according to the invention may vary. Preferably, the amount of enzymes, including catalase and SOD, are at least 10 weight % to about 40 weight % of the composition.

The composition is optimal if a number of different types of enzymes are present. Preferably, polysaccharidases are present in the composition in an amount of 5 to 25 weight %, proteases represent typically between 5 and 25 weight % in the composition and lipases represent between 3 and 15 weight % in the composition. Finally, antioxidant enzymes, preferably, represent maximal 15 weight % in the composition.

Vitamins and minerals are, preferably, present in an amount between 3 and 15 weight % of the composition. Glutamine is, preferably, present in an amount up to 20 weight % of the composition.

Complex forming agents and chelators can make up 40 weight % of the composition.

Further, other excipients such as dietary fibres, prebiotic fibres may be used as bulk material in the composition.

Despite their main function in the composition the different components of the composition may have multiple activities and have e.g. chelating, binding, antioxidant, antibacterial and/or anti-inflammatory activity.

A preferred composition according to the invention contains at least
polysaccharidases, proteases, lipases and/or antioxidant enzymes for decomposing a biofilm present in the GI tract,
lactoferrin as chelator for binding inorganic components such as iron,
Rosmarinus officinalis extract for its anti-adhesive effect on the bio film,
glutamine, vitamin A, vitamin D and zinc for facilitating recovery of mucosa cells of the gut, and
chitosan for an antibacterial activity against Gram-negative bacteria.

Preferably, a composition according to the invention is administered twice a day between the meals in an amount of about 2.5 to 3.0 gram. The composition may be administered in powder form, capsules, tablets and/or liquid or solid form. It could be incorporated in e.g. biscuits, in a biscuit filler or soup.

Tables 4a is an example of a typical composition according to a first embodiment of the invention. Tables 4b, 4c and 4d are examples of different compositions according to further embodiments of the invention.

TABLE 4a
Example of a typical composition according to a first embodiment of
the invention.

| | Amount | | |
|---|---|---|---|
| Component/Ingredient | (+/−mg/2.5 g) | | % weight |
| Vitamin B9 | 0.2 | mg | 0.008 |
| Vitamin B6 | 6 | mg | 0.255 |
| Vitamin C | 120 | mg | 5.091 |
| Vitamin B12 | 0.002 | mg | 0.00008 |
| Vitamin A | 0.6 | mg | 0.025 |
| Vitamin D | 0.4 | mg | 0.017 |
| Glutamine | 450 | mg | 19.09 |
| Zinc | 40 | mg | 1.697 |
| Magnesium | 200 | mg | 8.485 |
| Superoxide dismutase | 50 | mg | 2.121 |
| Catalase | 50 | mg | 2.121 |
| Polysaccharidases | 170 | mg | 7.212 |
| Protease complex | 100 | mg | 4.242 |
| Bromelain | 200 | mg | 8.485 |
| Lipase complex | 100 | mg | 4.242 |
| Curcuma longa | 80 | mg | 3.394 |
| Punica granatum extract | 120 | mg | 5.515 |
| Rosmarinus officinalis extract | 100 | mg | 4.242 |
| Citrus aurantium extract | 90 | mg | 3.818 |
| Chitosan | 130 | mg | 5.515 |
| Inositol | 50 | mg | 2.121 |
| Phosphatidylcholine | 50 | mg | 2.121 |
| Lactoferrin | 250 | mg | 10.606 |
| TOTAL | 2.357 | g | 100% |

Table 5 shows a specific test composition according to a fifth embodiment of the invention in which the composition contains four groups of ingredients (a), (b), (c) and (d). Ingredients of group (a) are intended to stimulate recovery of the intestinal epithelium and could be administered to patients for this purpose independently from the other groups of ingredients.

The test composition according to table 5 has been administered twice a day between the meals in an amount of 3.0 gram for a test treatment of 135 patients during a period of 90 days. All of the patients suffered from dysbiosis. The different ingredients were spread over different capsules for practical and stability reasons.

TABLE 4b
Example of a typical composition according to a second embodiment
of the invention.

| | Amount | | |
|---|---|---|---|
| Component/Ingredient | (+/−mg/3.1 g) | | % weight |
| Vitamin B9 | 0.2 | mg | 0.008 |
| Vitamin B6 | 6 | mg | 0.228 |
| Vitamin C | 120 | mg | 4.550 |
| Vitamin B12 | 0.002 | mg | 0.0001 |
| Vitamin A | 0.6 | mg | 0.023 |
| Vitamin D | 0.4 | mg | 0.015 |
| Glutamine | 450 | mg | 17.06 |
| Zinc | 40 | mg | 1.517 |
| Magnesium | 200 | mg | 7.584 |
| Superoxide dismutase | 50 | mg | 1.896 |
| Catalase | 50 | mg | 1.896 |
| Polysaccharidases | 170 | mg | 6.446 |
| Protease complex | 100 | mg | 3.792 |
| Bromelain | 200 | mg | 7.584 |
| Lipase complex | 100 | mg | 3.792 |
| Quercus rubra extract | 50 | mg | 1.896 |
| Quercus Petraea extract | 80 | mg | 3.034 |
| Rosmarinus officinalis extract | 100 | mg | 3.792 |
| Citrus aurantium extract | 90 | mg | 3.413 |
| Pistacia Lentiscus resin | 200 | mg | 7.584 |
| Chitosan | 130 | mg | 4.929 |
| Inositol | 50 | mg | 1.896 |
| Rice Bran | 150 | mg | 5.688 |
| Phosphatidylcholine | 50 | mg | 1.896 |
| Lactoferrin | 250 | mg | 9.480 |
| TOTAL | 2.637 | g | 100% |

TABLE 4c
Example of a typical composition according to a third embodiment of
the invention.

| | Amount | | |
|---|---|---|---|
| Component/Ingredient | (+/−mg/2.5 g) | | % weight |
| Vitamin B9 | 0.2 | mg | 0.008 |
| Vitamin B6 | 6 | mg | 0.239 |
| Vitamin C | 120 | mg | 4.786 |
| Vitamin B12 | 0.002 | mg | 0.0001 |
| Vitamin A | 0.6 | mg | 0.024 |
| Vitamin D | 0.4 | mg | 0.016 |
| Glutamine | 450 | mg | 17.953 |
| Zinc | 40 | mg | 1.596 |
| Magnesium | 200 | mg | 7.977 |
| Catalase | 50 | mg | 1.995 |
| Polysaccharidases | 170 | mg | 6.780 |
| Protease complex | 170 | mg | 6.780 |
| Lipase complex | 100 | mg | 3.989 |
| Quercus rubra extract | 50 | mg | 1.994 |
| Chitosan | 130 | mg | 5.185 |
| Rosmarinus officinalis extract | 100 | mg | 3.989 |
| Citrus aurantium extract | 90 | mg | 3.590 |
| Inositol | 50 | mg | 1.994 |
| Punica granatum extract | 100 | mg | 3.989 |
| Phosphatidylcholine | 50 | mg | 1.994 |
| Lactoferrin | 400 | mg | 15.954 |
| EDTA | 30 | mg | 1.197 |
| Active carbon | 200 | mg | 7.979 |
| TOTAL | 2.507 | g | 100% |

TABLE 4d
Example of a typical composition according to a fourth embodiment of
the invention.

| | Amount | | |
|---|---|---|---|
| Component/Ingredient | (+/−mg/2.5 g) | | % weight |
| Glutamine | 450 | mg | 25.6 |
| Vitamin A | 0.4 | mg | 0.0023 |
| Vitamin D | 0.4 | mg | 0.0023 |
| Zinc | 40 | mg | 2.3 |
| Magnesium | 200 | mg | 11.4 |
| Polysaccharidases | 170 | mg | 9.7 |
| Protease: Bromelain; Papain; Ficin | 100 | mg | 5.7 |
| Lipase complex | 100 | mg | 5.7 |
| Rosmarinus officinalis extract | 100 | mg | 5.7 |
| Quercus rubra extract | 120 | mg | 6.8 |
| Chitosan | 130 | mg | 7.4 |
| Inositol | 50 | mg | 2.8 |
| Lactoferrin | 250 | mg | 14.2 |
| Phosphatidylcholine | 50 | mg | 2.8 |
| TOTAL | 1.768 | g | 100% |

TABLE 5
Example of a specific test composition according to a fifth
embodiment of the invention, in which the composition contains
four groups of ingredients (a), (b), (c) and (d).

| | Amount | | |
|---|---|---|---|
| Component/Ingredient | (+/−mg/3.0 g) | | % weight |
| (a) +/−1000 mg | | | |
| Glutamine | 430 | mg | 14.326 |
| Curcuma longa extract | 100 | mg | 3.332 |
| Punica granatum extract | see below | | see below |
| Phosphatidylcholine | 50 | mg | 1.666 |
| Superoxyde Dismutase (SOD) | 50 | mg | 1.666 |
| Catalase | 50 | mg | 1.666 |
| Vitamin B6 | 10 | mg | 0.333 |
| Vitamin B9 | 300 | microgram | 0.010 |
| Vitamin B12 | 2 | microgram | 0.00007 |
| Vitamin A | 800 | microgram | 0.0267 |
| Vitamin C | 250 | mg | 8.329 |
| Vitamin D | 400 | microgram | 0.013 |
| Zinc | 60 | mg | 1.999 |
| (b) +/−800 mg | | | |
| complex of polysaccharidases: | 360 | mg | 11.994 |
| Alpha-Amylase; Beta-Amylase; | | | |
| Glucosamylase; Alpha- | | | |
| Galactosidase; Invertase; | | | |
| Maltase; Cellulase; | | | |
| Hémicellulase; Xylanase; | | | |
| Pectinase; Pectinesterase; | | | |
| Pullulanase; Dextranase | | | |
| protease complex: Bromelain; | 260 | mg | 8.662 |
| Papain; Ficin | | | |
| lipase, phospholipase | 180 | mg | 5.997 |
| (c) +/−700 mg | | | |
| Chitosan | 100 | mg | 3.331 |
| Punica granatum extract | 125 | mg | 4.165 |
| Rosmarinus officinalis extract | 125 | mg | 4.165 |
| Citrus aurantium extract | 80 | mg | 2.666 |
| Inositol | 50 | mg | 1.666 |
| Rice bran | 150 | mg | 4.998 |
| Magnesium | 70 | mg | 2.332 |
| (d) +/−500 mg | | | |
| Lactoferrin | 200 | mg | 6.663 |
| Activated carbon | 300 | mg | 9.995 |
| TOTAL | +/−3.0 | g | 100% |

Table 6 shows the results of bacterial analysis of stools of the set of test patients before and after the test treatment of 90 days. The values are mean values of the 135 patients with a standard deviation as indicated in the table 6.

TABLE 6
Bacterial analysis of stool during a test treatment in a set of 135 patients.

| | Time | | | |
|---|---|---|---|---|
| | Reference, typical | | N = 135/135* | N = 135/135 |
| | target values for | | 0 days | 90 days |
| | state of eubiosis | | Measured Mean (SD) | Measured Mean (SD) |
| Aerobic Flora | | | | |
| Escherichia coli | 5.10⁶-1.10⁷ | CFU/g** | (2.7 ± 0.2) × 10⁷ CFU/g | (5.4 ± 0.3) × 10⁵ CFU/g |
| Enterobacteriaceae | ≦9.1 0⁴ | CFU/g | (1.7 ± 0.2) × 10⁷ CFU/g | (6.2 ± 0.5) × 10⁴ CFU/g |
| Proteus mirabilis | ≦9.1 0⁴ | CFU/g | (9.6 ± 0.2) × 10⁴ CFU/g | (8.8 ± 0.2) × 10⁴ CFU/g |
| Proteus vulgaris | ≦9.1 0⁴ | CFU/g | (6.2 ± 0.3) × 10⁴ CFU/g | (7.6 ± 0.4) × 10⁴ CFU/g |
| Klebsiella oxytoca | ≦9.1 0⁴ | CFU/g | (4.9 ± 0.4) × 10⁶ CFU/g | (6.6 ± 0.3) × 10⁴ CFU/g |
| Klebsiella pneumoniae | ≦9.1 0⁴ | CFU/g | (6.8 ± 0.4) × 10⁴ CFU/g | (7.5 ± 0.3) × 10⁴ CFU/g |
| Citrobacter spp. | ≦9.1 0⁴ | CFU/g | (3.5 ± 0.4) × 10⁷ CFU/g | (7.5 ± 0.3) × 10⁴ CFU/g |
| Serratia spp. | ≦9.1 0⁴ | CFU/g | (5.9 ± 0.4) × 10⁴ CFU/g | (5.8 ± 0.2) × 10⁴ CFU/g |
| Hafnia alvei | ≦9.1 0⁴ | CFU/g | (9.0 ± 0.1) × 10⁴ CFU/g | (8.9 ± 0.2) × 10⁴ CFU/g |
| Morganella morganii | ≦9.1 0⁴ | CFU/g | (8.8 ± 0.2) × 10⁴ CFU/g | (8.7 ± 0.2) × 10⁴ CFU/g |
| Providencia spp. | ≦9.1 0⁴ | CFU/g | (8.7 ± 0.2) × 10⁴ CFU/g | (7.9 ± 0.5) × 10⁴ CFU/g |
| Pseudomonas spp. | ≦9.1 0⁴ | CFU/g | (6.9 ± 0.2) × 10⁴ CFU/g | (8.1 ± 0.4) × 10⁴ CFU/g |
| Enterococcus | 1.10⁶-1.10⁷ | CFU/g | (2.6 ± 0.3) × 10⁵ CFU/g | (1.4 ± 0.2) × 10⁶ CFU/g |
| Streptocoque β-hémolytique | ≦9.1 0⁴ | CFU/g | (1.1 ± 0.2) × 10⁵ CFU/g | (8.3 ± 0.2) × 10⁴ CFU/g |
| Bacillus spp. | ≦9.1 0⁴ | CFU/g | (7.6 ± 0.2) × 10⁴ CFU/g | (7.4 ± 0.5) × 10⁴ CFU/g |
| Staphylococcus aureus | ≦9.1 0⁴ | CFU/g | (8.1 ± 0.2) × 10⁴ CFU/g | (7.1 ± 0.5) × 10⁴ CFU/g |
| Anaerobic Flora | | | | |
| Bacteroides spp | 1.10⁸-1.10¹⁰ | CFU/g | (2.4 ± 0.2) × 10⁷ CFU/g | (1.3 ± 0.3) × 10⁹ CFU/g |
| Clostridium spp. | 1.10⁵ | CFU/g | (1.2 ± 0.3) × 10⁵ CFU/g | (1.1 ± 0.1) × 10⁵ CFU/g |
| Bifidobacterium spp. | 1.10⁸-1.10¹⁰ | CFU/g | (1.5 ± 0.2) × 10⁶ CFU/g | (1.6 ± 0.3) × 10⁹ CFU/g |
| Lactobacillus spp. | 1.10⁵-1.10⁸ | CFU/g | (3.2 ± 0.4) × 10⁴ CFU/g | (2.9 ± 0.3) × 1.10⁶ CFU/g |
| Salmonella spp. | Negative | | Negative | Negative |
| Yeast | | | | |
| Candida albicans | ≦1.10³ | CFU/g | (7.2 ± 0.4) × 10² CFU/g | (7.1 ± 0.2) × 10² CFU/g |
| Fungi | | | | |
| Geotrichum | ≦1.10³ | CFU/g | (2.2 ± 0.4) × 10³ CFU/g | (1.0 ± 0.1) × 10³ CFU/g |
| Mold | ≦1.10³ | CFU/g | (6.9 ± 0.4) × 10² CFU/g | (6.8 ± 0.3) × 10² CFU/g |

P < 0.01;
*N is the number of patients out of 135 tests;
**CFU/g, stool samples are collected and plated onto selective media to determine the amount of colony-forming unit (CFU) per gram of stool; the method and type of selective medium is known as such.

Initially, before the treatment, the flora shows an alteration of the balance of the intestinal flora indicating a state of dysbiosis. Resident flora, including e.g. Escherichia coli, Enterococcus spp., Bacteroides spp., Lactobacillus, Bifidobacterium, were underrepresented in all patients suffering from dysbiosis.

Before treatment, a significant colonization of potential pathogenic microorganisms, including Gram-negative and facultative anaerobic bacteria, in the colon has been observed.

After 90 days of treatment the results showed:
a strong decrease of pathogenic microorganisms in the composition of the colonic flora;
an increase of the resident beneficial microorganisms;
a balance in the transit flora and the resident flora; transit flora including e.g. Enterobacteriaceae, Enterococcus, Pseudomonas, Yersinia pestis, Klebsiella oxytoca, Proteus spp, Citrobacter spp,

Hence, it can be concluded that after treatment of 90 days the state of eubiosis has been restored in all patients.

The presence of virulence factors in stool of the 135 patient has also been analyzed before and after the test treatment. Results are shown in tables 7 and 8. These virulence factors are molecules expressed and secreted by pathogens, including bacteria, viruses, fungi and protozoa, that enable them to achieve the following (14-15):
immunoevasion, i.e. evasion of the host's immune response;
immunosuppression, i.e. inhibition of the host's immune response;
entry into and exit out of cells in case the pathogen is intracellular;
obtain nutrition from the host.

Extracellular enzymes secreted by pathogenic bacteria are considered to be one of the main types of virulence factors (16).

Hence, an increase of one or more virulent factors, which are produced by pathogenic bacteria, is an indication for intestinal dysbiosis.

Based on the results, patients are classified in 8 categories depending on the presence and the combination of indications of virulence. After 90 days of treatment for almost all patients the results of analyses regarding the virulence factors are negative. The results indicate the establishment of a balance of the intestinal microbiota, i.e. a state of eubiosis, and hence, a decrease of dysbiosis.

TABLE 7
Analysis of virulence factors (extracellular enzymes) during
a test treatment in a set of 135 patients, before start of the test treatment.

| | N = 85/135* | N = 15/135 | N = 11/135 | N = 8/135 | N = 8/135 | N = 5/135 | N = 2/135 | N = 1/135 |
|---|---|---|---|---|---|---|---|---|
| Catalase | + | + | + | + | − | + | + | − |
| Haemolysine | − | + | + | + | + | + | + | + |
| Coagulase | − | − | + | − | − | − | + | + |
| Urease | + | + | + | + | − | + | − | + |
| Gelatinase | − | − | + | − | − | + | + | + |
| Hyaloronidase | − | + | + | + | + | + | + | + |
| Collagenase | − | − | − | − | − | − | + | − |

*N is the number of patients out of 135 patients tested;
+, present, detected;
−, absent, not detected.

TABLE 8
Analysis of virulence factors (extracellular enzymes) during
a test treatment in a set of 135 patients, after 90 days of the test treatment.

| | N = 85/135* | N = 15/135 | N = 11/135 | N = 8/135 | N = 8/135 | N = 5/135 | N = 2/135 | N = 1/135 |
|---|---|---|---|---|---|---|---|---|
| Catalase | − | − | − | − | − | − | + | − |
| Haemolysine | − | − | − | − | − | − | − | + |
| Coagulase | − | − | − | − | − | − | − | − |
| Urease | − | − | − | − | − | − | − | − |
| Gelatinase | − | − | − | − | − | − | − | − |
| Hyaloronidase | − | − | − | − | − | − | − | + |
| Collagenase | − | − | − | − | − | − | − | − |

*N is the number of patients out of 135 patients tested;
+, present, detected;
−, absent, not detected.

Immunological markers in stool of the 135 patient has also been analyzed before and after the test treatment. Results are shown in table 9. Before the test treatment the intestinal immunity markers was very low, which confirms the weakness of intestinal immunity due to intestinal permeability and dysbiosis.

The results show that after the test treatment of 90 days using the test composition:
increase of secretory IgA (sIgA);
increase beta-defensin;
decrease activities of alpha-antitrypsinl;
decrease activities of the Calprotectin.

TABLE 9
Analysis of immunological markers in stool before and after a test
treatment in a set of 135 patients.

| | | N = 135/135* | N = 135/135 |
|---|---|---|---|
| | Time | 0 days | 90 days |
| | Reference | Mean (SD) | Mean (SD) |
| Intestinal | | | |
| immunity markers | | | |
| Secretory | 510-2040 | 277.5 ± 45 | 1997.4 ± 198 |
| Immunoglobulin | | | |
| A (sIgA ) | | | |
| Beta-defensin | <23.0 | 46.7 ± 8.3 | 22.3 ± 1.8 |
| Alpha-1- | <26.8 | 112.1 ± 5.9 | 11.4 ± 3.6 |
| antitrypsine | | | |
| Calprotectin | <50 | 185 ± 7.5 | 36 ± 5.9 |
| pH | 5.8-6.8 | 6.6-8.5 | 5.8-6.3 |
| Pancreatic | ≧200 μg/g | 359 ± 36.0 μg/g | 166 ± 12.1 μg/g |
| Elastase 1 | | | |

P < 0.01;
*N is the number of patients out of 135 patients tested.

Increase of the secretory IgA (sIgA) shows:
decrease of infection by antigens, endotoxins and certain proteins;
restoration of intestinal mucosa;
decrease of intestinal permeability;
decrease of dysbiosis;
increase of performance of the immunity of intestinal mucosa.

Human beta-defensins form an essential component of the intestinal lumen in innate immunity. Elevated human beta-defensin-2 levels indicate an activation of the innate immune system in patients especially with irritable bowel syndrome.

Decrease of the beta-defensin-2 shows:
decrease of infection by of bacteria pathogens;
reduction of inflammation at the level of the epithelium intestinal;
decrease in response to IL-1 or lipopolysaccharide (LPS);
increase in innate immunity;
balance of the intestinal microbiota, i.e. eubiosis;
decrease of intestinal permeability;
decrease of dysbiosis.

Elevated alpha-1-antitrypsin clearance suggests excessive gastrointestinal protein loss.

The fecal decrease of alpha-antitrypsin 1 shows:
decrease of permeability intestinal;
reduction of inflammation at the level of the epithelium intestinal;
balance of the intestinal microbiota, i.e. eubiosis;
decrease of dysbiosis.

The fecal calprotectin assay is a powerful marker of intestinal inflammation. It is significantly higher in patients with inflammatory bowel diseases (IBD). The decrease in fecal calprotectin shows:
strong decrease of inflammation at the level of the epithelium intestinal;
decrease of intestinal hyperpermeability;
balance of the intestinal microbiota, i.e. eubiosis;
decrease of dysbiosis.

The initial increased pH, i.e. before the test treatment, shows an alkalization of the colon. This is the result of a significant reduction of bacteria such as Lactobacillus and Bifidobacterium and a weak level of production of short chain fatty acids (SCFA). An increased pH allows the proliferation of pathogenic bacteria such as E. coli and Clostridium, etc. The decrease in faecal pH after the test treatment of 90 days shows:
proliferation bacteria such as Lactobacillus and Bifidobacterium;
production short chain fatty acids by fermenting bacteria, which are responsible for a decrease the intestinal pH;
inhibition of the proliferation of pathogenic microorganisms, decrease the production of ammonia, phenols and indols and a number of compounds sulphurous, regarded as harmful to health.

The determination of fecal elastase E1, commonly known as fecal elastase, aims to assess the proteolytic activity of fecal pancreatic origin. Unlike the chymotrypsin assay, it is not an immunoassay and a measure of proteolytic activity.

Hence, only if the immune system is massively supported, the disruption of the bio film has a lasting effect.

The combination of supporting the immune system, breaking down the bio films, putting pressure on harmful bacteria is, as experimental results show, giving back the strength of the immune system. Measurement in stools of sIgA, beta-defensin and fecal calprotectin proof that the bacterial flora is going back to the reference value and the inflammation of the gut returns to normal.

The present invention is not restricted to the compositions of the embodiments according to the invention as described above. From the description the synergistic function of different ingredients is made clear. Thus, according to the invention several ingredients listed in the compositions of the described embodiments may be combined in order to obtain further compositions, which are within the scope of the present invention. As such, for example, Pistacia lentiscus resin listed in the composition of second embodiment may be added to the compositions of the other embodiments; activated carbon may be added to the second embodiment.

### REFERENCES

1. Caroline Roper et al. (2010) “The role of lipopolysaccharides in virulence, biofilm, formation, and host specificity of Xylella Fastidiosa”. Dept. of Plant Pathol. & Microbiol. University of California Riverside.
2. Ce'cilia De Araujo et al. “Quorum sensing affects biofilm formation through lipopolysaccharide synthesis in Klebsiella pneumoniae”. Research in Microbiology. Volume 161, Issue 7, September 2010, Pages 595-603
3. Jason A. Hawrelak, BNat (Hons), PhD Candidate and Stephen P. Myers, PhD, BMed, ND, (2004) “The causes of Intestinal Dysbiosis: a Review”, Alternative Medicine Review. Volume 9, Number 2.
4. Ian W. Sutherland. (2001). “Biofilm exopolysaccharides: a strong and sticky framework”, Microbiology, 147, 3-9
5. Tamilvanan Shunmugaperumal. (2010). “Biofilm eradication and prevention, Pharmaceutical Approach to Medical Device Infections”, A JOHN WILEY & SONS, INC. PUBLICATION, 2010.
6. Medical Microbiology, 4th edition, Edited by Samuel Baron. Chapter 7 Bacterial Pathogenesis Johnny W. Peterson University of Texas Medical Branch at Galveston, Galveston, Tex. Galveston (Tex.): University of Texas Medical Branch at Galveston; 1996. ISBN-10: 0-9631172-1-1.
7. Jason A. Hawrelak, BNat (Hons), PhD Candidate and Stephen P. Myers, PhD, BMed, ND, “The causes of Intestinal Dysbiosis: a Review”, Alternative Medicine Review_Volume 9, Number 2_2004
8. Lewis, K. (2001). Riddle of biofilm resistance. Antimicrobial agents and chemotherapy, 45(4), 999-1007.
9. Shanmugam K. et al. (2008) “Plant-derived polyphenols attenuate lipopolysaccharide-induced nitric oxide and tumour necrosis factor production in murine microglia and macrophages”. Biochemistry and Molecular Biology, April; 52(4):427-38.
10. Horvath P J. 1981. The nutritional and ecological significance of acer-tannins and related polyphenols. MSc thesis, Cornell University, Ithaca, N.Y., USA.
11. H. Kadi, A. et al. (2011) “Antibacterial activity of ethanolic and aqueous extracts or Punica Granatum L. Bark”, Journal of applied Pharmaceutical Science, 01(10); 2011:180-182.
12. Chitosan as an antimicrobial compound: Modes of action and resistance mechanisms Dissertation zur Erlangung des Doktorgrades (Dr. rer. nat.) der MathematischNaturwissenschaftlichen Fakultät der Rheinischen FriedrichWilhelmsUniversität Bonnvorgelegt von Dina Raafat Gouda Fouad aus Alexandria/Ägypten; Bonn 2008.
13. Liu Hong-tao, et al. (2011) “Chitosan oligosaccharides suppress LPS-induced IL-8 expression in human umbilical vein endothelial cells through blockade of p38 and Akt protein kinases” Acta Pharmacol Sin. 2011 April; 32(4):478-86.
14. Arturo Casadevall and Liise-anne Pirofski, “Virulence factors and their mechanisms of action: the view from a damage—response framework”, Journal of Water and Health, 07.51, 2009.
15. Medical Microbiology, 4th edition, Edited by Samuel Baron. Chapter 7 Bacterial Pathogenesis Johnny W. Peterson University of Texas Medical Branch at Galveston, Galveston, Tex. Galveston (Tex.): University of Texas Medical Branch at Galveston; 1996.
16. Salmond G. P. C (1994). “Secretion of Extracellular Virulence Factors by Plant Pathogenic Bacteria”. Annual Review of Phytopathology Vol. 32: 181-200.

1. A pharmaceutical composition for use in the treatment of chronic inflammation of the gastrointestinal tract, containing a pharmaceutically effective amount of at least
(i) enzymes selected from the group of polysaccharidases, proteases, lipases and/or antioxidant enzymes, capable of decomposing a biofilm that is present in the gut;
(ii) at least one chelator, comprising lactoferrin, for binding inorganic components such as iron; and
(iii) at least one binder, capable of binding organic components originating from the decomposition of the biofilm and/or bacteria;
(iv) glutamine, vitamin A, vitamin D and zinc, for facilitating recovery of mucosa cells of the gut.

2. Composition for use according to claim 1, further comprising chitosan, for an antibacterial activity against Gram-negative bacteria.

3. Composition for use according to claim 1, further comprising Rosmarinus officinalis extract, for reducing activity and adhesion of the biofilm.

4. Composition for use according to claim 1, wherein the binder comprises at least one binder capable of binding organic components originating from the decomposition of the biofilm and at least one binder capable of binding organic components originating from the decomposition of bacteria of the biofilm.

5. Composition for use according to claim 1, wherein the binder comprises at least one binder for binding organic components originating from the decomposition of the biofilm selected from the group of inositol and/or rice bran or a combination thereof.

6. Composition for use according to claim 1, wherein the binder comprises at least one binder for binding organic components originating from the decomposition of bacteria of the biofilm selected from the group of Punica granatum extract, Rosmarinus officinalis extract, Quercus rubra extract, Quercus petraea extract, phosphatidylcholine and/or magnesium or a combination thereof.

7. Composition for use according to claim 6, wherein a first binder is selected from the group of Punica granatum extract, Quercus rubra extract and/or Quercus petraea extract or a combination thereof and a second binder is selected from the group of phosphatidylcholine and/or magnesium or a combination thereof.

8. Composition for use according to claim 1, further containing at least one anti-inflammatory ingredient selected from the group of Curcuma longa extract, Punica granatum extract, Citrus aurantium extract, Quercus extract, superoxide dismutase and/or catalase or a combination thereof.

9. Composition for use according to claim 1, further containing vitamin B9, vitamin B6, vitamin C, vitamin B12 and/or magnesium, in order to facilitate recovery of mucosa cells of the gut.

10. Composition for use according to claim 1, further containing activated carbon for absorbing and eliminating toxins in stool.

11. Composition for use according to claim 1, further containing Pistacia lentiscus resin.

12. Composition for use according to claim 1, wherein said polysaccharidases are selected from the group of alpha-amylase, beta-amylase, glucosamylase, alpha-galactosidase, invertase, maltase, cellulase, hemicellulase, xylanase, pectinase, pectinesterase, pullulanase and/or dextranase or a combination thereof.

13. Composition for use according to claim 1, wherein said proteases are selected from the group of bromelain, papain and/or ficin or a combination thereof.

14. Composition for use according to claim 1, wherein said lipases comprise phospholipase.

15. Composition for use according to claim 1, containing at least
10-40 weight % of enzymes,
3-15 weight % of vitamins and dietary minerals,
1-20 weight % of glutamine,
1-40 weight % of complex forming agents and chelators,
0-50 weight % of a bulk material,
the total of the composition being 100 weight %.

16. Composition for use according to claim 1, comprising at least polysaccharidase, protease and lipase.

17. Composition for use according to claim 1, containing said enzymes in an amount of
5-25 weight % of polysaccharidases,
5-25 weight % of proteases,
3-15 weight % of lipases,
0-15 weight % of antioxidant enzymes,
the total of the composition being 100 weight %.

18. Food supplement for use in the treatment of chronic inflammation of the gastrointestinal tract containing a composition according to claim 1.

19. Functional food for use in the treatment of chronic inflammation of the gastrointestinal tract containing a composition according to claim 1.

20. A composition according to claim 1 for use in treatment of dysbiosis in the gastrointestinal tract.
